SEQUENCE LISTING (16 sequences)

SEQ ID NO: 1 - DNA, 63 bp, Artificial
  synthetic oligonucleotide
  misc_feature (37)..(43): n is a, c, g, or t
  misc_feature (63)..(63): n is a, c, g, or t
ccatctcatccctgcgtgtctccgactcagcgtactnnnnnnnttttttttttttttttttvn

SEQ ID NO: 2 - DNA, 7 bp, Artificial
  synthetic oligonucleotide
  misc_feature (1)..(7): n is a, c, g, or t
nnnnnnn

SEQ ID NO: 3 - DNA, 6 bp, Artificial
  synthetic oligonucleotide
cgtact

SEQ ID NO: 4 - DNA, 30 bp, Artificial
  synthetic oligonucleotide
ccatctcatccctgcgtgtctccgactcag

SEQ ID NO: 5 - DNA, 19 bp, Artificial
  synthetic oligonucleotide
agatgtgtataagagacag

SEQ ID NO: 6 - DNA, 14 bp, Artificial
  synthetic oligonucleotide
tcgtcggcagcgtc

SEQ ID NO: 7 - DNA, 15 bp, Artificial
  synthetic oligonucleotide
gtctcgtgggctcgg

SEQ ID NO: 8 - DNA, 58 bp, Artificial
  synthetic oligonucleotide
aatgatacggcgaccaccgagatctacactctttccctacacgacgctcttccgatct

SEQ ID NO: 9 - DNA, 58 bp, Artificial
  synthetic oligonucleotide
caagcagaagacggcatacgagatgtgactggagttcagacgtgtgctcttccgatct

SEQ ID NO: 10 - DNA, 5 bp, Artificial
  synthetic oligonucleotide
cgata

SEQ ID NO: 11 - DNA, 29 bp, Artificial
  synthetic oligonucleotide
aatgatacggcgaccaccgagatctacac

SEQ ID NO: 12 - DNA, 24 bp, Artificial
  synthetic oligonucleotide
caagcagaagacggcatacgagat

SEQ ID NO: 13 - DNA, 9 bp, Artificial
  synthetic oligonucleotide
  misc_feature (1)..(9): n is a, c, g, or t
nnnnnnnnn

SEQ ID NO: 14 - DNA, 35 bp, Artificial
  synthetic oligonucleotide
  misc_feature (1)..(1): n is 5′-phosphorylated adenine
  misc_feature (35)..(35): n is 3′-dideoxycytidine
ngcaacgcactttgaattttgtaatcctgaagggn

SEQ ID NO: 15 - DNA, 34 bp, Artificial
  synthetic oligonucleotide
cccttcaggattacaaaattcaaagtgcgttgct

SEQ ID NO: 16 - DNA, 27 bp, Artificial
  synthetic oligonucleotide
  misc_feature (27)..(27): n is a, c, g, or t
aaaaaaaaaaaaaaaaaaaaaaaaabn

(19) United States
(12) Patent Application Publication
(10) Pub. No.: US 2020/0385712 A1
(43) Pub. Date: Dec. 10, 2020

(54) METHOD FOR COMPREHENSIVELY ANALYZING 3' END GENE EXPRESSION OF SINGLE CELL

(71) Applicant: HITACHI, LTD., Tokyo (JP)

(72) Inventors: Kiyomi TANIGUCHI, Tokyo (JP); Masataka SHIRAI, Tokyo (JP)

(73) Assignee: HITACHI, LTD., Tokyo (JP)

(21) Appl. No.: 16/769,434

(22) PCT Filed: Nov. 9, 2018
(86) PCT No.: PCT/JP2018/041702

§ 371 Date: Jun. 3, 2020

(30) Foreign Application Priority Data
Dec. 11, 2017 (JP) 2017-236839

(51) Int. Cl. C12N 15/10; C12Q 1/6837

(52) U.S. Cl. CPC C12N 15/1096; C12Q 1/6837

(57) ABSTRACT

A method for analyzing gene expression in a cell using a device, the method including: introducing a plurality of cells into microreactors so that a single cell corresponds to each microreactor, capturing mRNA from the single cell on the probe, synthesizing first cDNA by subjecting the captured mRNA to a reverse transcription reaction, to produce a first cDNA library derived from the single cell on the solid supports, washing the solid supports, a step of synthesizing second cDNA from the first cDNA library, performing fragmentation of double-stranded DNA containing the first and second cDNA and addition of a tag sequence, removing a component other than an immobilized double-stranded DNA fragment by washing the solid supports with a washing solution, performing amplification of the double-stranded DNA fragment, and performing, for the amplified sequence, analysis of gene expression in each single cell, using the cell identification sequence and the molecule identification sequence.

### TECHNICAL FIELD

The present invention relates to a system, a method, and a kit for comprehensively analyzing gene expression at a single-cell level using a 3′ sequence of mRNA.

### BACKGROUND ART

In an analysis method using a tissue or a plurality of cells as a sample for analysis (a bulk analysis method), measurement is generally performed by averaging differences between cells, and therefore, integrated analysis of a biological activity of each cell and between cells is difficult to be performed. Particularly, in immune system cells, nerve cells, and pluripotent stem cells, types of genes that are expressed and expression levels thereof are greatly different in each cell, and development of a technique for analyzing gene expression at a single-cell level has rapidly progressed for the purpose of clarifying a mechanism of a disease.

In particular, by remarkable technological development of a reagent related to amplification and Next Generation Sequencer (NGS) since 2005, development of a technique for a comprehensive gene expression analysis to examine all genes has been accelerated, and learning expression statuses of a vast number of genes in detail has become possible. In particular, in a basic research field such as regenerative medicine and clarification of a disease mechanism, in which a search for a novel marker gene is important, needs for the comprehensive gene expression analysis technique have been high.

Hitherto, as a means for analyzing comprehensive gene expression, a method in which sequencing is performed on full-length first cDNA (entire gene) synthesized from mRNA (RNA-seq) has been mainstream. In general, an apparatus and a reagent for sequencing are extremely costly in NGS analysis. Meanwhile, since a length (size) of decoded bases in each run of the NGS analysis is limited, a cost of sequencing required for each detected gene is high in the RNA-seq method, which may be problematic. Therefore, a method for performing the NGS analysis by preparing only a sample of first cDNA corresponding to a 3′ end sequence of mRNA, by which a cost can be decreased to about one fifth to one tenth, has also been prevailing in recent years.

For example, in a research related to the clarification of a disease mechanism, statistical understanding of information on individual cells in a cell mass forming a tissue related to the disease is important. The greater a size of the cell mass is, the greater the amount of obtained information is, which is advantageous. For this reason, in recent years, the number of cells to be analyzed is required to be increased to several thousands to 10,000 or more, and accordingly, labor (complication) and a reagent cost (including a cost for the NGS analysis) for preparing a DNA library sample for the NGS analysis from each one of the cells are problems that should be overcome. Development of a technique for synthesizing first cDNA from a single cell by separating many cells into individual reactors one by one has recently progressed, and a device using a cell sorter, a microchannel, and a droplet is prevailing. For example, Soumillon et al. (NPL 1) have succeeded in analyzing gene expression in a total of 12,832 cells by performing NGS analysis by amplifying a DNA sequence derived from a 3′ end sequence of mRNA after sorting the cells in a microwell plate using a cell sorter. In the method, forty-four 384-well plates are consumed, and the cells are sorted into each one of the wells. Then, several microliters of a reverse transcription reaction reagent are added to each well while dispensing a probe for a reverse transcription reaction having sequences different for each well (for cell identification), thereby synthesizing first cDNA. A problem arises here in that each reagent must be dispensed into a total of 16,896 wells, which requires intensive labor, and a cost for the reagent is extremely high, since a massive amount of the reagent, at least several tens to 100 mL, is consumed (NPL 1).

Furthermore, a technical problem that should be overcome still exists regarding a detection rate (detection sensitivity) and quantification precision in a low-expression gene group, since the amount of mRNA contained in a single cell is a trace amount, which is about 0.5 pg (10⁵ to 10⁶ molecules).

As an approach to solve such problems, PTL 1 discloses a method for analyzing expression of a plurality of genes with high precision, even in a case of a low-expression gene of which the amount is about 10 copies per cell, the method including, for the purpose of performing highly precise quantitative analysis by preventing a sample loss from a trace amount of mRNA, for example, first capturing mRNA derived from a single cell with high efficiency on a surface of a magnetic bead on which a large number of probes are immobilized, and quantitatively analyzing a cDNA library sample thus synthesized by a real-time PCR method. Furthermore, PTL 2 discloses a method for analyzing gene expression with a number of cells at a single-cell level using a chip formed of a porous membrane or beads arranged in a two-dimensional array. That is, since a probe having a cell identification sequence that is different for each region where each of the cells is captured is immobilized on a support, a cell identification sequence different for each cell can be introduced into a cDNA library thus synthesized. Since a number of cells can be analyzed at a single-cell level through parallel processing by collectively performing NGS analysis on the obtained samples, the complication and the reagent cost for the sample preparation can be reduced to one hundredth or less.

### CITATION LIST

### Patent Literature

PTL 1: US 2012/0245053 A

PTL 2: US 2016/0010078 A

### Non-Patent Literature

NPL 1: Soumillon et al., bioRxiv, Internet homepage https://doi.org/10.1101/003236, 2014

### SUMMARY OF INVENTION

### Technical Problem

In order to realize comprehensive gene expression analysis with high accuracy at a single-cell level, it is important to perform the comprehensive gene expression analysis on (i) 1,000 to 10,000 cells for analysis and with (ii) high detection sensitivity and quantification precision. In addition, a cost for the analysis needs to be low in terms of a practical use.

While development of a technique for increasing the number of cells for analysis in (i) has progressed, problems still remain regarding (ii). Specifically, in a method for preparing a sample for the comprehensive gene expression analysis, steps as many as 10 or more in total are needed to be undergone in general (for example, (1) a step of sorting single cells into a microreactor, (2) a cell lysis step, (3) an mRNA capturing step, (4) a first cDNA synthesis step, (5) a second cDNA synthesis step, (6) a first PCR amplification step, (7) a purification step, (8) a step of fragmenting DNA by an enzyme treatment, (9) a step of ligating a tag sequence for second PCR amplification (mostly including an index for sample identification), (10) a purification step, (11) a second PCR amplification step using the added sequence, (12) a purification step, and (13) a DNA quantification step). Therefore, it is important how many of DNA molecules derived from an initial sample of a trace amount of mRNA molecules, which is about 0.5 pg (10⁵ to 10⁶ molecules) per single cell, are caused to remain in a final sample by proceeding the sample preparation while maintaining high reaction efficiency in each of the series of steps. Particularly, it is important that a sample loss is avoided in the first half of the steps up to the PCR amplification. Furthermore, in sample preparation using a trace amount of DNA such as that in single-cell analysis, target DNA is easily fragmented and degraded into short fragments of 250 bases or less, in a case where an optimum reaction condition (the amount of the enzyme, reaction time, and a temperature) in the step of fragmenting DNA by an enzyme treatment (including a tagmentation step) in particular is even slightly shifted, and becomes a large factor for the sample loss, which may be problematic. In general, a commercially available enzyme reagent for the DNA fragmentation step requires at least 1 to 100 ng of DNA, and the activity thereof is so strong that, when this amount is converted to the number of cells (cDNA derived from mRNA), it corresponds to at least several thousands to 10⁵ cells. In general, the fragmentation reaction is difficult to be immediately and completely stopped, and even during the several tens of seconds or several minutes of performing an operation for proceeding to the next step, degradation of a target molecule proceeds, resulting in a sample loss, which may be problematic.

In addition, in the PCR step after the DNA fragmentation (tagmentation) step, amplification of a by-product (a sequence not having a molecule identification sequence, a cell identification sequence, or a sequence for amplification) cannot be completely prevented, and there is no means for solely amplifying only a DNA region derived from a 3′ end of target mRNA. In other words, due to the presence of the by-product, each component required for amplification such as a DNA polymerase, dNTP, and a primer is used in a reaction with the by-product, and amplification efficiency of a target DNA decreases, thus decreasing a proportion of amplified target DNA molecules.

That is, the most important issue in performing the comprehensive gene expression analysis with high accuracy and high precision at a low cost is whether or not a target DNA sample derived from a 3′ end sequence of mRNA can be prepared under a reaction condition that maximizes utilization efficiency of an initial sample of a trace amount of mRNA molecules in a single cell by avoiding a “sample loss” in the initial sample caused in the course of several reaction steps.

The present invention has been made in view of such problems, and an object of the present invention is to provide a method for efficiently performing the comprehensive gene expression analysis at a single-cell level.

### Solution to Problem

As a result of conducting various studies for solving the above problems, the present inventors developed a method of preparing a final sample in which utilization efficiency of an initial sample of mRNA molecules in a single cell is maintained to be high when simultaneously performing the comprehensive gene expression analysis on a plurality of cells at a single-cell level, by which comprehensive gene expression data can be acquired with high accuracy.

In one aspect, the present invention provides a method for analyzing gene expression in a cell using a device including a plurality of microreactors, for example, a device into which a plurality of chips (or arrays) are incorporated in parallel, wherein the microreactors are filled with one or more solid supports on which a probe having a primer sequence for amplification, a cell identification sequence, a molecule identification sequence, and an oligo (dT) sequence is immobilized, and the method includes: a step of introducing a plurality of cells into the microreactors so that a single cell corresponds to each one of the microreactors; a step of capturing mRNA derived from the single cell on the probe; a step of synthesizing first cDNA by subjecting the captured mRNA to a reverse transcription reaction, to produce a first cDNA library derived from the single cell on the solid supports; a step of (pooling and) washing the solid supports; a step of synthesizing second cDNA from the first cDNA library; a step of performing fragmentation of double-stranded DNA containing the first cDNA and the second cDNA and addition of a tag sequence; a step of removing a component other than an immobilized double-stranded DNA fragment by washing the solid supports with a washing solution; a step of performing amplification of the double-stranded DNA fragment using a primer having at least a portion of the primer sequence for amplification and the tag sequence or a sequence complementary to at least a portion of the primer sequence for amplification and the tag sequence to amplify only a sequence derived from a 3′ end sequence of the mRNA; and a step of performing, for the amplified sequence, analysis of gene expression in each single cell, using the cell identification sequence and the molecule identification sequence.

Additional features related to the present invention will become clear from the description of the present specification and the attached drawings.

### Advantageous Effects of Invention

According to the present invention, since the gene expression analysis is simultaneously and collectively performed on a plurality of cells, by amplifying only the sequence derived from the 3′ end of mRNA, labor and a reagent cost for the analysis can be reduced. Furthermore, a target DNA molecule derived from mRNA is retained on a solid support (magnetic bead) throughout many steps in the entire method, a first cDNA synthesis step, a second cDNA synthesis step, a tagmentation step, and a PCR step, and therefore, a residual reagent can be completely removed simply by washing using the magnetic bead in each of the steps, and all of the target DNA molecules derived from the mRNA can be collected with 100% of efficiency. That is, a reaction can proceed with high efficiency using an optimum reagent condition in each of the steps, and a sample loss does not occur during purification. Therefore, a final sample, which is obtained from a trace amount of mRNA molecules in a single cell by allowing a reaction to proceed in a state in which utilization efficiency is maximized and amplifying only a sequence derived from a 3′ end of the mRNA, can be applied in NGS analysis, and comprehensive gene expression data can be acquired with high accuracy and high precision. The present invention can be applied to drug discovery, clarification of mechanisms in various diseases, regenerative medicine, and the like, and can contribute to development of life science.

### BRIEF DESCRIPTION OF DRAWINGS

FIG. 1-1A is a plan view of an example of a chip in which microreactors are arranged in an array. FIG. 1-1B is a cross-sectional view of an example of the chip; an enlarged view FIG. 1-1C illustrates an example of a step of capturing mRNA eluted after cell lysis in the microreactor; and a further enlarged view FIG. 1-1D is a schematic view illustrating an example of a step of synthesizing first cDNA on a surface of a support (magnetic bead).

FIG. 1-2 is a schematic view illustrating an example of each of steps after synthesizing the first DNA using the chip up to preparing a DNA library, which is a final sample, and performing NGS analysis.

FIG. 2A illustrates a schematic view of another example of a method for synthesizing second cDNA on the support using a random primer and a strand-displacement DNA polymerase. FIG. 2B illustrates a schematic view of yet another example of the method for synthesizing second cDNA on the support using a single-stranded DNA ligase. FIG. 2C illustrates a schematic view of yet another example of the method for synthesizing second cDNA on the support using a terminal transferase.

FIG. 3A is an example of a schematic view illustrating a step of synthesizing first cDNA on a surface of a support (magnetic bead) on which two types of probes, a probe 109 for a reverse transcription reaction (SEQ ID NO: 1) and a random primer 213 for second cDNA synthesis, are immobilized. FIG. 3B is an example of a schematic view of a method for synthesizing second cDNA on a support using an immobilized random primer 213 and the strand-displacement DNA polymerase.

FIG. 4 is a schematic view of two types of tag sequences that are added.

FIG. 5 is a graph showing amounts of amplified DNA products obtained by PCR amplification using a sample washed after a tagmentation reaction (Example 1) and a sample to which a neutralizing solution provided in a commercially available kit is added.

FIG. 6 is a graph showing the comparison of amounts of DNA contained in (1) a PCR amplification product sample obtained by using a forward primer having a sequence 112 for PCR amplification and a reverse primer having a common sequence portion 121 of 19 bases, (2) a PCR amplification sample obtained by using a forward primer having the sequence 112 for PCR amplification and a reverse primer having both a specific sequence A portion (14 bases) and a specific sequence B portion (15 bases), and (3) a PCR amplification sample obtained by using a forward primer having the sequence 112 for PCR amplification, a reverse primer having both the specific sequence A portion (14 bases) and the specific sequence B portion (15 bases), and a primer (P5) having a sequence for NGS (SEQ ID NO: 11) and a primer (P7) having a sequence for NGS for supporting the amplification, each of which uses the same sample as a template that is obtained by the tagmentation reaction and includes target DNA immobilized on a support.

FIG. 7 is a graph showing experimental data obtained by analyzing ERCC (Ambion, a sample obtained by mixing known amounts of 92 types of mRNA) according to a method described in Example 1, for the purpose of investigating quantification precision.

FIG. 8 is a graph showing experimental data of single-cell analysis performed by the method described in Example 1.

FIG. 9 is a graph showing an average number of detected genes per cell and a total number of detected genes per chip (here, 100 kinds of cell identification tags) obtained by the method described in Example 1.

### DESCRIPTION OF EMBODIMENTS

The present invention relates to a method for performing comprehensive gene expression analysis on a plurality of cells simultaneously at a single-cell level. Specifically, a plurality of cells are simultaneously captured using a device into which a plurality of chips (or arrays) that include a plurality of microreactors arranged therein in an array are incorporated in parallel, mRNA derived from a single cell are captured with high efficiency, and then, a first cDNA is synthesized. It may be preferable that the first cDNAs derived from the plurality of cells are pooled in one tube, and a residual reagent is washed away. Subsequently, a second cDNA is synthesized in the tube, and then after a tagmentation reaction (or a reaction of adding a tag sequence by carrying out a reaction of fragmenting double-stranded DNA using a DNA fragmentation enzyme and then further carrying out a ligation reaction), unnecessary fragmentated DNAs may be removed by performing washing with a surfactant containing a tagmentation inhibitor, thereby efficiently performing PCR amplification of only a 3′ end portion of the mRNA. In a final sample obtained by purifying the amplification product, a great amount of DNA obtained by avoiding a “sample loss” during various reaction steps to maximize utilization efficiency of an initial sample (3′ end of mRNA in each cell) may be contained.

In the present specification, the term “gene expression analysis” means quantitatively analyzing a gene in a sample (a cell, a tissue section, or the like), that is, expression of mRNA, analyzing expression distribution of a gene (mRNA) in a sample, obtaining correlation data between a specific cell or position in a sample and a gene (mRNA) expression level, and the like. A sample may not be particularly limited as long as it is a sample derived from a living body which is desired to be subjected to gene expression analysis, and any sample such as a cell sample, a tissue sample, and a liquid sample can be used. In addition, the living body from which a sample is derived may also not be particularly limited. In the present specification, a DNA fragment derived from a 3′ end of mRNA to be analyzed may be collectively referred to as a “target DNA”.

In the present specification, the term “comprehensive gene expression analysis” means performing parallel expression analysis of a plurality of genes contained in a cell, and examples thereof can include performing parallel expression analysis of at least 1,000 or more genes. In addition, the term “gene expression analysis at a single-cell level” means performing expression analysis of a gene (mRNA) contained in a single cell, which is distinguished from analyzing an average expression level of genes contained in a plurality of cells.

In one aspect, the present disclosure provides a method for analyzing gene expression in a cell using a device including a plurality of microreactors, for example, a device into which a plurality of chips (or arrays) are incorporated in parallel, wherein the microreactors are filled with one or more solid supports on which a probe having a primer sequence for amplification, a cell identification sequence, a molecule identification sequence, and an oligo (dT) sequence is immobilized, and the method includes: a step of introducing a plurality of cells into the microreactors so that a single cell corresponds to each one of the microreactors, a step of capturing mRNA derived from the single cell on the probe, a step of synthesizing first cDNA by subjecting the captured mRNA to a reverse transcription reaction, to produce a first cDNA library derived from the single cell on the solid supports, a step of (pooling and) washing the solid supports, a step of synthesizing second cDNA from the first cDNA library, a step of performing fragmentation of double-stranded DNA containing the first cDNA and the second cDNA and addition of a tag sequence, a step of removing a component other than an immobilized double-stranded DNA fragment by washing the solid supports with a washing solution, a step of performing amplification of the double-stranded DNA fragment using a primer having at least a portion of the primer sequence for amplification and the tag sequence or a sequence complementary to at least a portion of the primer sequence for amplification and the tag sequence to amplify only a sequence derived from a 3′ end sequence of the mRNA, and a step of performing, for the amplified sequence, analysis of gene expression in each single cell, using the cell identification sequence and the molecule identification sequence.

The device including the plurality of microreactors may be a device into which a plurality of chips, so-called two-dimensional arrays, configured to analyze gene expression are incorporated in parallel, and the microreactors in the device may be filled with one or more solid supports on which the probe having the primer sequence for amplification, the cell identification sequence, the molecule identification sequence, and the oligo (dT) sequence is immobilized. Such device is known in the art and may not be particularly limited. For example, devices described in PTL 1, PTL 2, WO 2016/038670 A, and the like can be used.

The solid support which fills the microreactor may preferably be produced using a material having a large surface area in order to increase mRNA capturing efficiency. For example, one or more beads, a porous structure, a mesh structure, and the like may preferably be adopted. In a case where the bead is used as the solid support, the bead can be produced with a resin material (polystyrene or the like), an oxide (glass or the like), a metal (iron or the like), sepharose, a combination thereof, and the like. Due to ease of operation, a magnetic bead (a paramagnetic bead) may preferably be used. The solid support having a size of 10 nm to 100 μm in diameter, for example, the bead having a size of 10 nm to 100 μm in diameter may be preferable. Furthermore, a microporous sheet, a porous film, or the like may also be provided so that the solid support does not escape from the microreactor.

The probe having the primer sequence for amplification, the cell identification sequence, the molecule identification sequence, and the oligo (dT) sequence is immobilized on the solid support, and such probe can be synthesized by a conventional oligonucleotide synthesis method and can be immobilized on the solid support by any method known in the art. A polymerization degree of oligo (dT) may be a polymerization degree that can allow hybridization of the oligo (dT) with a poly A sequence of mRNA, thus allowing capturing of mRNA on the solid support on which the oligo (dT) is immobilized. The polymerization degree can be, for example, about 10 to 20 bases. By introducing the primer sequence for amplification into the probe, the sequence can be used as a common primer in an amplification step (for example, PCR). Furthermore, as the cell identification sequence, a cell identification sequence having a known sequence which is different for each microreactor may be used. For example, in a case where a random sequence of 5 bases is used, 4⁵=1,024 positions or regions can be identified. In other words, analysis can be performed while identifying mRNA (target DNA) derived from each of 1,024 single cells in one operation. Furthermore, as the molecule identification sequence, a molecule identification sequence having a random sequence different for each probe molecule (an mRNA molecule, or a DNA molecule derived from mRNA) may be used. In a case where the molecule identification sequence (for example, of 7 bases) is introduced into the probe, 4⁷=1.6×10⁵ molecules can be recognized, and therefore, it is possible to recognize which molecule amplification products having a sequence of the same gene that is derived based on the same cell are each derived from, from a sequence data of amplification products obtained by Next Generation Sequencer (NGS). That is, since an amplification bias can be corrected using the molecule identification sequence, highly precise quantification data can be obtained. The cell identification sequence and the molecule identification sequence are described in detail in, for example, WO 2014/141386 A.

Here, in order to subsequently synthesize the second cDNA, a DNA probe having a random primer may be further immobilized on the solid support. The random primer may not be particularly limited as long as the random primer has a length and composition that allows the random primer to function as a primer, and for example, a random primer having a length of 6 to 15 bases can be used.

One through-hole may be formed in each microreactor, and the single cell may be captured in the through-hole. The through-hole can be suitably set according to a size of a cell to be analyzed, and a diameter of the through-hole may preferably be 10 μm or smaller.

Using the device, the plurality of cells may be introduced into the microreactors in a manner that a single cell corresponds to each one of the microreactors. At this time, the single cell may be captured in each through-hole by applying a negative pressure (suction) to the through-hole. Whether capturing of the cell in the through-hole has been performed or not may be confirmed by using an observation device as necessary, and the cell may be reintroduced into the through-hole as necessary. Since a cell that is not captured may affect a subsequent step, the cell may preferably be removed by, for example, introduing and discharging of a washing solution.

Next, mRNA derived from the single cell may be captured on the probe immobilized on the solid support. In the present invention, the expression “capturing of mRNA” means extracting an mRNA molecule contained in a cell and separating the molecule from other cellular components. Specifically, a cell lysate known in the art may be dispensed into the microreactors, and mRNA may be extracted from each of the captured single cells. For example, a cell may be lysed using a proteolytic enzyme, a chaotropic salt such as guanidine thiocyanate and guanidine hydrochloride, a surfactant such as Tween and SDS, or a commercially available cell lysis reagent (for example, a Lysis solution), and a nucleic acid contained in the cell, that is mRNA, can be eluted. A status of the cell lysis may be confirmed by using an observation device, as necessary. The eluted mRNA may be captured on the probe by binding to the oligo (dT) sequence of the probe.

The device, the microreactor, and the solid support may be washed using a washing solution as necessary, thereby removing an unnecessary component and reagent.

Next, the first cDNA having a sequence complementary to the mRNA sequence or to a portion of the mRNA sequence may be synthesized by subjecting the captured mRNA to a reverse transcription reaction. The synthesis of the first cDNA, that is synthesis of a complementary strand, can be performed by a method known in the art. For example, cDNA can be synthesized by carrying out a reverse transcription reaction using a conventional reverse transcriptase or a reverse transcriptase having a template switch function. After the synthesis reaction, the mRNA may be removed by degradation using, for example, RNase. As a result, a cDNA library consisting of the first cDNA corresponding to the mRNA may be produced on the solid support. Since a single cell corresponds to one microreactor, the first cDNA library derived from the single cell can be produced on the solid support contained in each of the microreactors.

Thereafter, a residual reagent, for example, the reverse transcriptase, a deoxyribonuclease, or the like, can be removed by performing the step of washing the solid support (the first cDNA library), and then the step of synthesizing the second cDNA and the amplification step can be performed without being impeded.

Before or after the step of washing the solid support, a step of pooling the solid supports, on which the first cDNA libraries derived from the single cells are immobilized, corresponding to the plurality of cells may be performed. For example, the solid supports, on which the first cDNA libraries derived from the single cells and produced in each of the plurality of microreactors in one chip are immobilized, may be collectively put into one tube, and the first cDNA libraries corresponding to the plurality of cells can be pooled. For example, the first cDNA libraries corresponding to about 100 to 10,000 cells can be pooled per chip. Therefore, subsequent steps can be collectively performed on the first cDNA libraries derived from the single cells corresponding to the plurality of cells, and thus, simplification of operation and reduction of a reagent cost can be achieved. Since the cell identification sequence is present on the solid support as described above, even in a case of mixing and pooling the first cDNA libraries corresponding to the plurality of cells, it is possible to identify which of the microreactors (which of the single cells) the first cDNA library is derived from when performing the gene expression analysis. Furthermore, since a plurality of chips are incorporated into one device in parallel, it is possible to process 1,600 to 160,000 cells per one reaction by using, for example, a device into which 16 chips are incorporated. A chip identification tag may also be introduced into the finally prepared sample during the PCR amplification step, and therefore, even in a case where samples derived from all of the cells are pooled into one sample and subjected to analysis by the next generation sequencer, the gene expression analysis can be performed by distinguishing the cells.

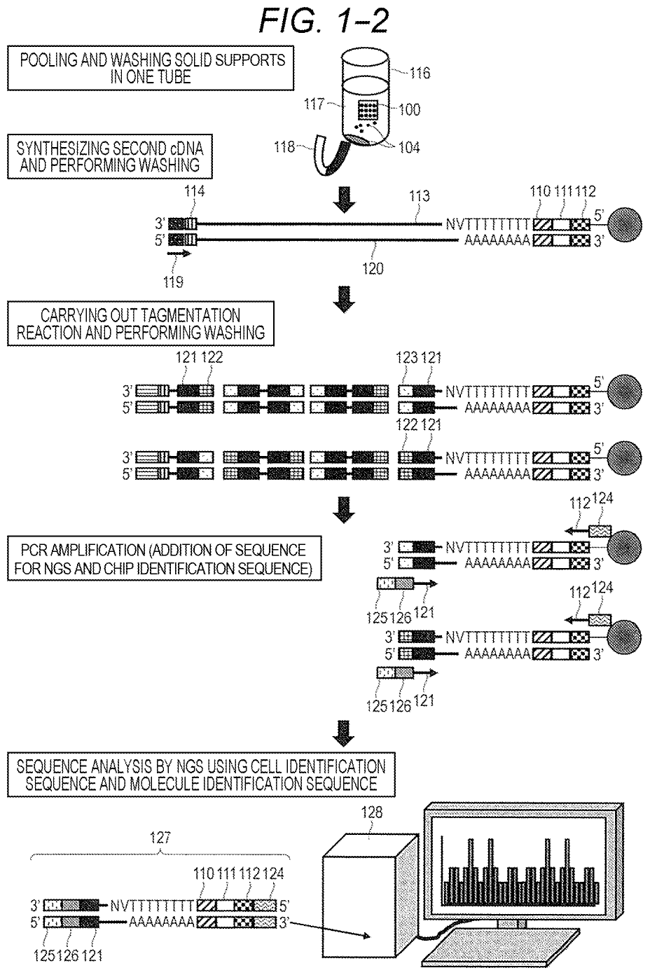

Next, the second cDNA may be synthesized from the first cDNA library. The step of synthesizing the second cDNA can be performed using a complementary strand synthesis reaction known in the art. Several examples will be shown, and those skilled in the art can select and carry out a suitable method. One method is synthesizing the second cDNA by a complementary strand elongation reaction using a random primer and a DNA polymerase having a strand displacement activity. The random primer may not be particularly limited as long as the random primer has a length and composition that allows the random primer to function as a primer, and for example, a random primer having a length of 6 to 15 bases can be used. The DNA polymerase having a strand displacement activity is also known in the art, and for example, Phi29 DNA polymerase, Bst DNA polymerase, Csa DNA polymerase, and the like are commercially available. By using this method, a reaction may be caused to take place as shown in FIG. 2A, for example, and the second cDNA can be synthesized with high synthesis efficiency.

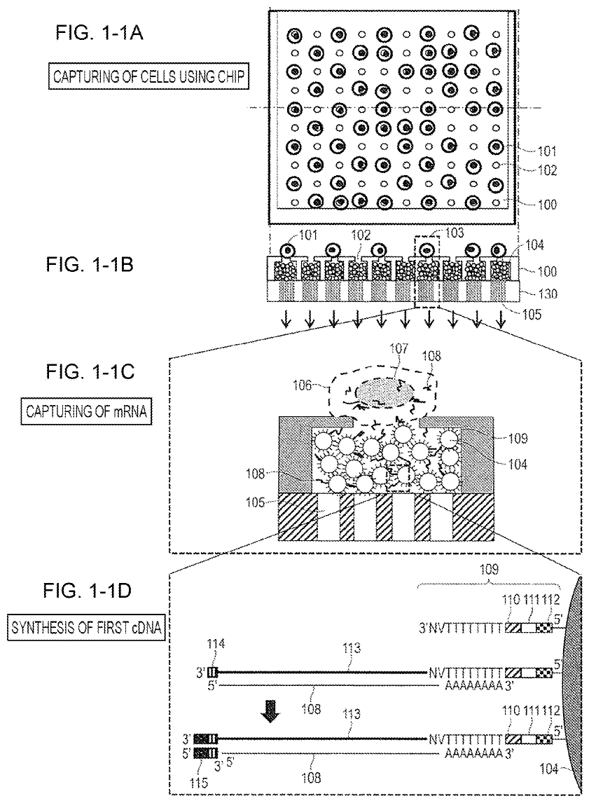

Another example of the step of synthesizing the second cDNA is using a specific sequence, since the specific sequence is added to the first cDNA in a case where a reverse transcriptase having a template switch function is used when synthesizing the first cDNA. For example, SmartScribe Reverse Transcriptase, SuperScript II, SuperScript IV, and the like are commercially available. That is, the second cDNA may be synthesized by a complementary strand elongation reaction using a primer having a sequence complementary to the added specific sequence. As a DNA polymerase, a conventional DNA polymerase can be used. For example, Tks Gflex DNA polymerase, Ex Hot start DNA Polymerase, Platinum Taq DNA Polymerase High Fidelity, and the like are commercially available. By using this method, a reaction may be caused to take place as shown in “SYNTHESIZING SECOND cDNA AND PERFORMING WASHING” of FIG. 1-2, for example, and the second cDNA can be synthesized.

Yet another example of the step of synthesizing the second cDNA is synthesizing the second cDNA by first adding a known sequence to the 3′ end of the first cDNA using a single-stranded DNA ligase and carrying out a complementary strand elongation reaction using a primer having a sequence complementary to the known sequence. As the single-stranded DNA ligase, for example, Circ Ligase ssDNA Ligase and the like are commercially available. In addition, a length and a composition of the known sequence to be added can also be suitably set. For example, a sequence having a length of 10 to 30 bases can be added. By using this method, a reaction may be caused to take place as shown in FIG. 2B, for example, and the second cDNA can be synthesized.

Yet another example of the step of synthesizing the second cDNA is synthesizing the second cDNA by first adding a polyN sequence (a poly T, A, G, or C sequence) to the 3′ end of the first cDNA using a terminal transferase (TdT) and carrying out a complementary strand elongation reaction using a primer having a sequence complementary to the polyN sequence. As the terminal transferase (TdT) and the DNA polymerase, conventional terminal transferase and DNA polymerase can be used, and those skilled in the art can select and use suitable terminal transferase and DNA polymerase. In addition, a type and a length of the polyN sequence to be added can also be suitably set. For example, a polyN sequence having a length of 10 to 30 bases can be used. By using this method, a reaction may be caused to take place as shown in FIG. 2C, for example, and the second cDNA can be synthesized.

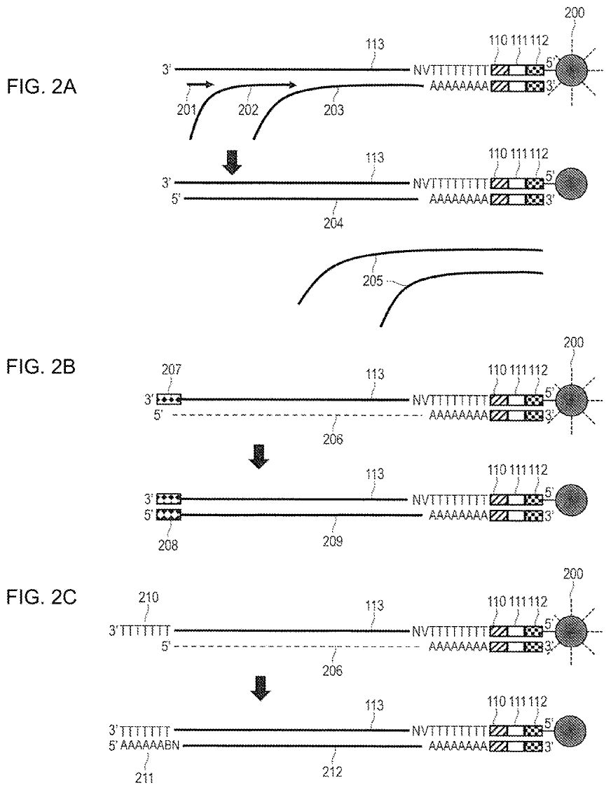

Another embodiment of the step of synthesizing the second cDNA may include immobilizing a DNA probe having a random primer on the solid support in advance, synthesizing the second cDNA in the step of synthesizing the second cDNA by a complementary strand elongation reaction using the random primer immobilized on the solid support and a DNA polymerase having a strand displacement activity, thereby amplifying the cDNA. The DNA polymerase having a strand displacement activity is as described above, and any DNA polymerase can be used. By using this method, a reaction may be caused to take place as shown in FIG. 3B, for example, thereby synthesizing the second cDNA and further amplifying cDNA. Therefore, sensitivity can be increased by amplification, even in a case of using mRNA of which only a trace amount is present. Furthermore, for the purpose of increasing efficiency of the second cDNA synthesis reaction and the amplification reaction, the reactions may proceed both in a liquid phase and on the solid support by adding the random primer (not immobilized, FIG. 2A) in a reaction liquid phase as well.

After the second cDNA is synthesized, the fragmentation of double-stranded DNA containing the cDNA and the second cDNA, and the addition of a tag sequence may be performed. As one method, a tagmentation reaction can be used. The tagmentation reaction is a reaction of fragmenting double-stranded DNA and adding a tag sequence, and is a reaction known in the art. An enzyme (transposase) and a reagent that can be used are commercially available, and those skilled in the art can perform the tagmentation reaction by using suitable enzyme and reagent. As another method, a reaction of fragmenting the double-stranded DNA may be performed by using a DNA fragmentation enzyme, and then a reaction of adding the tag sequence may be performed by carrying out a ligation reaction. The DNA fragmentation enzyme and an enzyme (ligase) used in the ligation are also known in the art, and those skilled in the art can select a suitable reagent. The tag sequence to be added may not be particularly limited as long as the tag sequence has a length and composition suitable for a primer to bind in the subsequent amplification step. For example, the tag sequence can be a nucleotide sequence having a length of about 20 to 35 bases.

Next, a component other than the immobilized double-stranded DNA fragment may be removed by washing the solid supports with the washing solution. Activities of the enzymes used for the DNA fragmentation and the tag addition in the previous step, in particular, the enzyme used for the tagmentation reaction (transposase), can be immediately stopped, thereby reducing effects thereof on a subsequent step. For example, it may be preferable that the solid support is washed with a washing solution having an inhibitory effect on the enzyme that is used. By the washing step, only target DNA (that is, a sequence derived from the 3′ end of mRNA) having a short length of several hundreds of bases may be extracted, and DNA of another sequence, which is a by-product, can be removed. In other words, reduction in a cost, labor, and analysis time in gene identification (sequencing) and quantitative analysis can be achieved in a subsequent gene expression analysis step, compared to a general case of using a full-length DNA sequence.

Only a sequence derived from the 3′ end sequence of the mRNA may be amplified by performing amplification of the double-stranded DNA fragment using the primer having at least a portion of the primer sequence for amplification and the tag sequence or a sequence complementary to at least a portion of the primer sequence for amplification and the tag sequence. Another sequence may be added to the primer. For example, a sequence for identifying the chip used or a sequence required for subsequent NGS analysis may be added to the primer. Design of the primer, an amplification reaction condition, and the like may be known in the art and can be suitably selected according to a length of an amplification target sequence, a reagent used, and the like. Furthermore, after producing the first cDNA library on the solid support and carrying out various reactions (the step of synthesizing the first cDNA, the step of synthesizing the second cDNA, and the tagmentation step), a residual reagent and a by-product can be simply and completely removed by washing, and therefore, only the target DNA, which is the sequence derived from the 3′ end sequence of the mRNA, can be obtained in a state of being immobilized on the solid support, without a sample loss. Since a sample obtained by subjecting the target DNA to PCR amplification only contains the target DNA prepared from a trace amount of mRNA molecules derived from each cell by maximizing utilization efficiency, a favorable result can be obtained in final gene expression analysis.

Next, analysis of gene expression in a single cell may be performed on the amplified sequence using the cell identification sequence and the molecule identification sequence. Specifically, the amplified sequence may be provided for sequencing by next generation sequencer (NGS), and gene expression in a single cell may be analyzed. Since the amplified sequence has the chip identification sequence, the cell identification sequence, and the molecule identification sequence, it is possible to analyze gene expression by identifying which chip the sequence is derived from, which single cell the sequence is derived from, and which molecule the sequence is derived from, using these sequences as indexes.

The gene expression analysis method of the present disclosure described above can be easily and simply carried out by using a kit including the device, the reagents such as an enzyme, the washing solution, and/or a disposable vessel (a tube) that are necessary for performing each step, and/or instructions including a description for carrying out a relevant method, and the like.

Furthermore, the gene expression analysis method of the present disclosure can be easily and simply carried out using a system including the device into which the plurality of chips are incorporated, means for introducing the reagent, the washing solution, and the like, means for observing the chip, means for applying a negative pressure (suction) to the chip, and the like that are necessary for performing each step.

Hereinafter, the present invention is more specifically described using Examples. Note that the Examples below are for providing examples of the method for analyzing comprehensive gene expression at a single-cell level and are not intended to limit the present invention.

### EXAMPLE 1

The method for comprehensively analyzing gene expression at a single-cell level includes, as illustrated in flows shown in FIG. 1-1A-D and FIG. 1-2, (1) a step of capturing cells on a chip in which microreactors are arranged in an array, (2) a step of capturing mRNA after cell lysis, (3) a step of synthesizing first cDNA on a surface of a support, (4) a step of pooling and washing the supports (magnetic beads) on which first cDNA libraries are immobilized by dispersing the supports in one tube, (5) a step of synthesizing second cDNA and performing washing, (6) a step of carrying out a tagmentation reaction and performing washing, (7) a PCR amplification step, and (8) an NGS analysis step. Depending on cases, the step (4) can be automatically performed in a device equipped with a chip, and the steps prior to the PCR amplification (the reaction steps (1), (2), (3), (4), (5), and (6)) can be collectively performed using the device, in a state of retaining DNA on the supports. By using the method of the present Example, labor in various steps of preparing a sample from each of a plurality of cells simultaneously can be reduced. Hereinafter, each of the steps is described in detail.

### (1) Step of Capturing Cells on Chip

A device equipped with sixteen chips 100 in which a hundred microreactors 103 are arranged in an array (FIG. 1-1A a plan view and) FIG. 1-1B a cross-sectional view) is prepared. Each of the microreactor 103 is filled with an ample amount of supports (preferably magnetic beads) 104 on which probes 109 for a reverse transcription reaction (SEQ ID NO: 1) consisting of a sequence 112 for PCR amplification (SEQ ID NO: 4), a cell identification sequence 111 (SEQ ID NO: 3) which is a known sequence of 6 bases that is different for each microreactor, a molecule identification sequence 110 (SEQ ID NO: 2) consisting of a random sequence of 7 bases which is different for each probe molecule, and a sequence of oligo (dT) VN are immobilized with high density. A micro through-hole 102 having a diameter (2 to 6 μm) that is smaller than that of a cell is present on an upper surface of the microreactor, and a reagent discharge part 105 through which a reagent passes while the supports are retained in the microreactor by adhesion of a porous membrane (pore diameter: 0.8 μm, Merck Millipore) 130 is present on a lower surface of the microreactor. That is, since the device used in the present Example has a structure in which a negative pressure can be applied from a lower side of the chip, a reagent can be discharged from the inside and an upper part of the microreactor by passing through the reagent discharge part 105 of the microreactor. The microreactors 103 are washed by, first, adding 2 μL of phosphate buffered saline (PBS) containing an RNase inhibitor (1 U/μL) to upper surfaces of all of the chips 100 equipped in the device and then discharging PBS by applying a negative pressure. A cell suspension is prepared by diluting human colon cancer cells (HCT116) expressing a green fluorescent protein (GFP) with PBS to 100 cells/μL, 0.8 μL of the cell suspension containing about 80 cells is added to the upper surfaces of all of the chips, and a negative pressure is immediately applied thereto. PBS is thus discharged through lower surfaces of the chips (FIG. 1-1B), and cells 101 sufficiently larger than the micro through-hole 102 are captured on the upper surfaces of the microreactors 103.

The plurality of cells 101 (the number of injected cells in the present Example: 1,280) can be simultaneously captured on the upper surfaces of the microreactors. The capturing of the cells is completed within about 1 minute, which is confirmed by observation using a fluorescence microscope. Since a position of the microreactor can be specified by using the cell identification sequence 111 as a lead after output of NGS analysis data, which is a final result, it is possible to investigate the size and the state of the cell by comparing the position with a moving image and an image of the capturing of the cell.

(2) Step of Capturing mRNA on Support

2 μL of a cell lysis reagent (100 mM Tris (pH 8.0), 500 mM NaCl, 10 mM EDTA, 1% SDS, and 5 mM DTT) is added to the upper surfaces of all of the chips, and the chips are incubated at a room temperature for 2 minutes while applying a weak negative pressure. By this operation, a cell membrane 106 (FIG. 1-1C) and a nuclear membrane 107 are lysed, and mRNA 108 contained in each cell is eluted in the microreactor. Since a poly A sequence is present on the 3′ end of mRNA, an mRNA molecule is captured with an oligo (dT) sequence portion on a 3′ end side of the probe 109 for a reverse transcription reaction (FIG. 1-1D). For example, since the number of the probes 109 for a reverse transcription reaction immobilized on each support 104 having a diameter of 1 μm is 5×10⁴ to 10⁵ molecules, and the number of supports that fills each microreactor is 10⁵ to 2×10⁵, the number of the probes for a reverse transcription reaction per microreactor is 5×10⁹ to 2×10¹⁰ molecules. That is, the amount of the probes is sufficient for capturing 10⁵ to 10⁶ molecules of mRNA contained in a single cell, and the mRNA can be captured with high efficiency.

(3) Step of Synthesizing First cDNA on Surface of Support

The cell lysis reagent is completely removed by increasing the negative pressure in the device. 2 μL of a cell washing solution (100 mM Tris (pH 8.0), 500 mM NaCl, and 5 mM DTT) is further added to the upper surfaces of all of the chips, and a negative pressure is immediately applied. Each microreactor is thoroughly washed by performing this operation twice, thereby removing the cell lysis reagent that can be an inhibitor for a subsequent reverse transcription reaction. 4 μL of a reverse transcription reaction reagent (1x lysis buffer, 1x Ultra Low First Strand Buffer, SMART-Seq v4 Oligo 115 (3.6 μM), SMART Scribe RT (13.8 U/μL), and RNase Inhibitor (1.5 U/μL): Takara Bio Inc.) is added to the upper surfaces of all of the chips, the microreactors are filled with the reagent by applying a moderate negative pressure, and then the chips are incubated at 42° C. for 90 minutes. First cDNA 113 is thus synthesized in a 3′ direction of the probe 109 for a reverse transcription reaction, by using a captured mRNA molecule 108 as a template. Since the reverse transcriptase SMART Scribe RT used in the present Example has a template switch (TS) function, a specific sequence 114 of several bases is added to a 3′ end of the synthesized first cDNA. Next, the SMART-Seq v4 Oligo 115 having a sequence complementary to the specific sequence 114 on the 3′ end side thereof complementarily binds to the specific sequence 114, and the synthesis of the first cDNA further proceeds by using the SMART-Seq v4 Oligo 115 as a template. Accordingly, the finally synthesized first cDNA has a sequence complementary to the SMART-Seq v4 Oligo 115 on a 3′ end side thereof and the sequence 112 for PCR amplification (SEQ ID NO: 4), the cell identification sequence 111 (SEQ ID NO: 3), and the molecule identification sequence 110 (SEQ ID NO: 2) on a 5′ end side thereof (FIG. 1-1D). Through this step, first cDNA libraries can be simultaneously synthesized from the mRNA derived from all of genes that are expressed in a plurality of single cells, in a state of being immobilized on the supports.

(4) Step of Pooling and Washing Supports on Which First cDNA Libraries are Immobilized in one Tube

The reverse transcription reaction solution is removed by slightly increasing the negative pressure in the device, and the chip 100 and the porous membrane 130 which acts as the reagent discharge part 105 while retaining the supports on the lower surface of the chip at the same time are taken out using a tweezer and put into 20 μL of a buffer 117 for dispersing supports (50 mM Tris, 50 mM NaCl, and 0.1% Tween 20, pH 8.0) in a tube 116. A material for the chip (PDMS or the like) has low intrinsic fluorescence and flexibility, and a material for the porous membrane also has flexibility. Therefore, the supports 104 that fill the microreactor are easily dispersed in the buffer 117 by shaking or rubbing the chip in the buffer using a tweezer while placing a neodymium magnet 118 at a bottom part of the tube. Therefore, first cDNA library samples simultaneously synthesized from each of the plurality of cells using the chip are pooled. The cell identification sequence 111 is different for each of the first cDNA libraries (microreactors), and therefore, the pooling performed in this step is not problematic, since each of the first cDNA libraries from each cell can be distinguished in the NGS analysis data. Furthermore, the larger the number of the pooled cells are, the more labor and cost required for sample preparation can be reduced. Dispersing of all of the supports into the buffer is visually confirmed, and the chip and the porous membrane are removed from the tube. The buffer in which a residual reverse transcription reaction reagent or the like is solubilized is removed while the supports 104 are captured by the neodymium magnet 118, and after washing with 50 μL of a support washing solution (10 mM Tris and 0.1% Tween 20 (pH 8.0)), the supports are suspended in 1 μL of 10 mM Tris (pH 8.0).

(5) Step of Synthesizing Second cDNA and Performing Washing

5 μL of a second cDNA synthesis reagent (1x Tks Gflex Buffer, Tks Gflex DNA polymerase (0.125 U/μL), and a primer 119 for second cDNA synthesis (0.72 μM): Takara Bio Inc.) is mixed with the supports in the same tube, and a reaction is carried out using a thermal cycler with the following temperature condition: 98° C. for 1 minute, 58° C. for 5 minutes, and 68° C. for 6 minutes, thereby synthesizing second cDNA 120. A supernatant which contains a residual reagent from the second cDNA synthesis reaction is removed while the supports are captured by the neodymium magnet 118, and the supports are washed with 50 μL of a support washing solution (10 mM Tris and 0.1% Tween 20 (pH 8.0)).

### (6) Step of Carrying out Tagmentation Reaction and Performing Washing

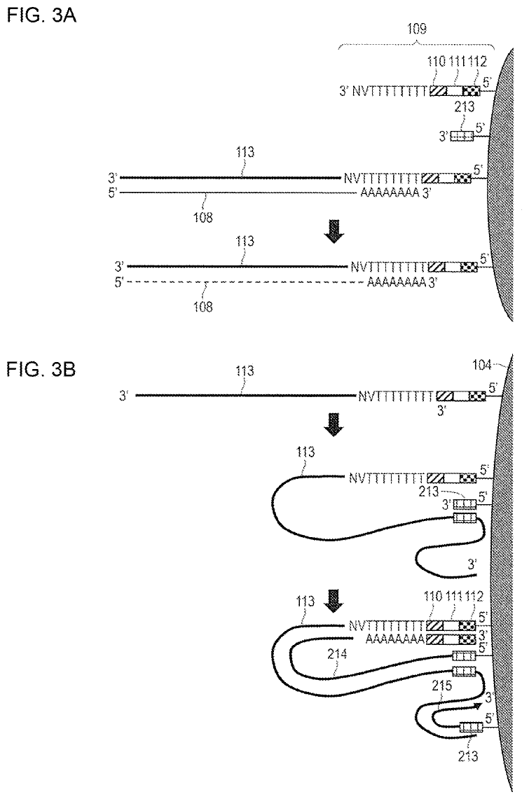

1 μL of a tagmentation reagent (a mixture solution consisting of 0.25 μL of sterile water, 0.5 ∥L of Amplicon Tagment Mix, and 0.25 μL of Tagment DNA buffer: Illumina, Inc.) is mixed with the supports, and the mixture is incubated at 55° C. for 2.5 minutes, and then the temperature is decreased to 10° C. Double-stranded DNA which is in a state of being retained on the supports is fragmented into fragments of 250 to 1,000 bases or less, and at the same time, two types of tag sequences (FIG. 4), a tag sequence A consisting of a common sequence portion 121 (SEQ ID NO: 5) and a specific sequence A portion 122 (SEQ ID NO: 6) and a tag sequence B consisting of the common sequence portion 121 and a specific sequence B portion 123 (SEQ ID NO: 7), are randomly added by a transposase contained in the tagmentation reagent. Immediately after completion of the reaction, 50 μL of a surfactant washing solution (0.1% Tween 20, 100 mM Tris (pH 8.0), and 500 mM NaCl) which contains a high concentration of salt and has been cooled on ice is added to the mixture. A supernatant containing a residual reagent from the tagmentation reaction is removed while capturing the supports by the neodymium magnet 118. After washing the supports by repeating this operation twice, the supports are washed twice in the same manner with 50 μL of a washing solution (0.1% Tween 20 and 20 mM Tris (pH 8.0)) which has been cooled on ice. Since activity of the transposase can be stopped by immediately and completely removing the transposase, and DNA fragments that are not retained on the supports (by-products in the tagmentation reaction) can be completely removed as well by this operation, only the target DNA (having a sequence derived from a 3′ end of the mRNA) can be obtained in a state of being retained on the supports. Here, any one of the tag sequence A and the tag sequence B is added to the fragmented DNA which is in a state of being retained on the supports.

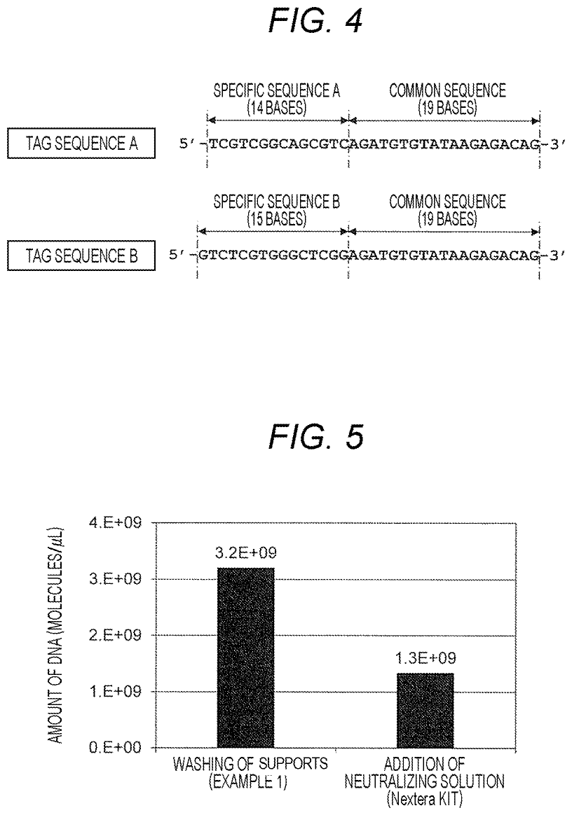

In addition, in a general tagmentation reaction (Illumina, Inc.), reaction activity is decreased by adding a neutralizing solution and performing incubation for 5 minutes. In this conventional method, the reaction activity is difficult to be completely stopped, and even during the short period of proceeding to the next step (several tens of seconds or several minutes), the target DNA is degraded into short fragments (200 to 250 or less) due to excessive DNA fragmentation activity, resulting in a sample loss, which may be problematic. FIG. 5 is experimental data obtained by comparing amounts of DNA after performing PCR amplification using a sample obtained by applying the washing of the supports according to the method of the present Example and a sample obtained by using the neutralizing solution according to the conventional method. Compared to the conventional method (right graph) which is affected by a sample loss due to fragmentation, the amount of DNA obtained according to the method of the present Example (left graph) is confirmed to be 2.5 times larger than that in the conventional method. In particular, in a case of subjecting a trace amount of DNA to a reaction such as the single-cell analysis, the problem of the sample loss is serious, since the sample loss largely affects detection sensitivity and quantification precision. However, such problem can be avoided according to the method of the present Example.

### (7) PCR Amplification Step

For the purpose of activating a DNA polymerase in advance, 28.6 μL of a reaction solution (1x Tks Gflex Buffer and Tks Gflex DNA polymerase (0.025 U/μL): Takara Bio Inc.) is incubated at 98° C. for 1 minute, and then cooled to 4° C. This reaction solution is mixed with 1 μL of a forward primer (10 μM) obtained by adding a sequence 124 for NGS analysis (P5_R1SP) (SEQ ID NO: 8) to a 5′ side of the sequence 112 for PCR amplification (SEQ ID NO: 4) and 0.4 μL of a reverse primer obtained by adding a chip identification sequence 126 (SEQ ID NO: 10) and a sequence 125 for NGS analysis (P7_R2SP) (SEQ ID NO: 9) to a 5′ side of the common sequence portion 121 (SEQ ID NO: 5) to prepare 30 μL of a PCR reaction solution. The PCR reaction solution is mixed with the sample after the tagmentation reaction on ice. Next, the mixture is incubated at 68° C. for 30 seconds and 98° C. for 45 seconds, and 14 cycles of PCR amplification consisting of 98° C. for 15 seconds, 60° C. for 45 seconds, and 68° C. for 30 seconds. The resultant is then cooled to 4° C. are performed using a thermal cycler. About 30 μL of a PCR amplification product sample, which is a supernatant, is collected in a separate tube using a neodymium magnet. A residual PCR product is additionally collected by washing the surfaces of the supports and an inner wall of the tube with 20 μL of 0.1% Tween 20 (10 mM Tris (pH 8.0)) and then mixed with the PCR amplification product sample (50 μL in total). A DNA sample is purified and quantified using Ampure XP beads, thereby obtaining a final sample 127 for performing NGS analysis. In the PCR amplification step, the chip identification sequence 126 which is a known sequence of 5 bases that is different for each chip (tube) is introduced into the target DNA. That is, since each of the 16 chips used in the present Example can be identified, it is theoretically possible to distinguish a total of 1,600 cells by combining the result of the chip identification with 100 kinds of the cell identification sequences 111. Therefore, the comprehensive gene expression analysis can be performed on 1,600 cells by performing NGS analysis once.

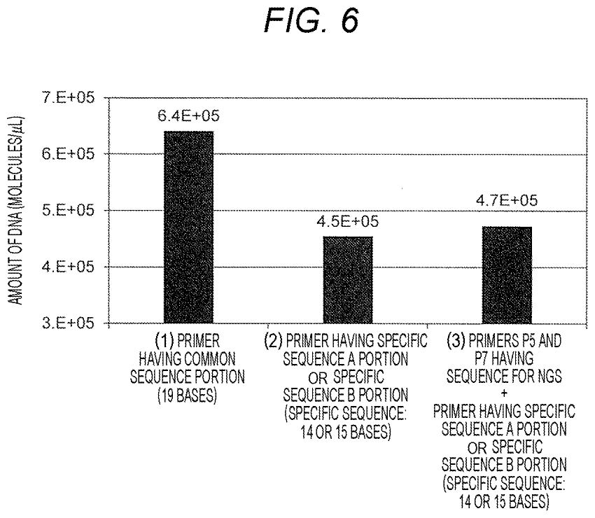

Furthermore, in the method of the present Example, the DNA immobilized on the support (the DNA sequence derived from the 3′ end of the mRNA), to which any one of the tag sequence A and the tag sequence B is added, obtained after the tagmentation reaction is used as a template, and a reverse primer having the common sequence portion 121 (SEQ ID NO: 5) of 19 bases included in both of the tag sequences (FIG. 4) is used. On the other hand, in the conventional method, primers using the specific sequence A portion (14 bases) (FIG. 4) and the specific sequence B portion (15 bases) (FIG. 4) are used, and a binding strength between complementary strands of the target DNA and the primer is weak, since a sequence of the primer that complementarily binds to the template is as short as 14 or 15 bases. FIG. 6 is experimental data obtained by comparing amounts of DNA contained in (1) a PCR amplification product sample obtained by using a forward primer having the sequence 112 for PCR amplification (SEQ ID NO: 4) and a reverse primer having the common sequence portion 121 of 19 bases, (2) a PCR amplification sample obtained by using a forward primer having the sequence 112 for PCR amplification and a reverse primer having both the specific sequence A portion (14 bases) and the specific sequence B portion (15 bases), and (3) a PCR amplification sample obtained by using a forward primer having the sequence 112 for PCR amplification, a reverse primer having both the specific sequence A portion (14 bases) and the specific sequence B portion (15 bases), and a primer (P5) having a sequence for NGS (SEQ ID NO: 11) and a primer (P7) having a sequence for NGS (SEQ ID NO: 12) for supporting the amplification, each of which uses the same sample as a template that is obtained by the tagmentation reaction and includes target DNA immobilized on a support. The amount of DNA in the sample of (1), which is the method of the present Example, can be confirmed to be significantly large. That is, it is considered that a length of a complementary strand binding sequence in (1), which is the method of the present Example, is as long as 19 bases, and thus the sequence can more stably anneal to the template, and the PCR amplification can be performed with higher efficiency, compared to those in (2) and (3), which are conventional methods in which complementary strand binding sequences are as short as 14 or 15 bases. Furthermore, it is considered that, since the target DNA (the DNA sequence derived from the 3′ end of the mRNA) serving as the template is generally not immobilized on the support, and a by-product in the tagmentation reaction (a by-product produced by addition of the two types of tags to a DNA fragment derived from a portion of the mRNA other than the 3′ end thereof) remains in the sample, target DNA amplification efficiency is further reduced by consumption of the DNA polymerases and primers in amplification of the by-product in the PCR amplification step. In addition, a PCR amplification product derived from the by-product may have an adverse effect on quantification precision and sensitivity in NGS analysis. Therefore, various problems in a conventional amplification process can be avoided in the method of the present Example.

### (8) Step of Performing NGS Analysis

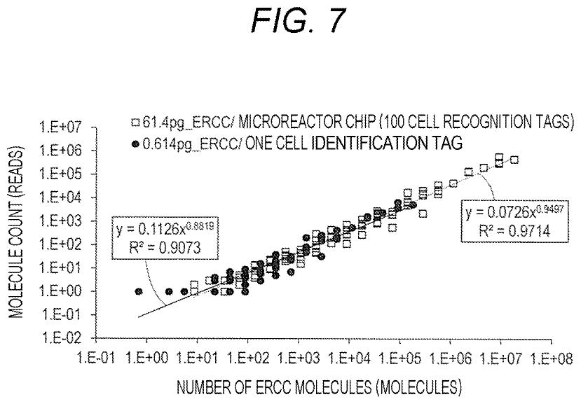

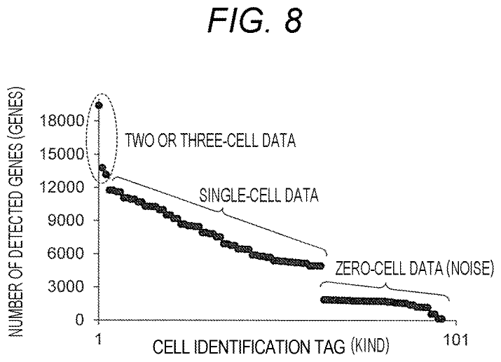

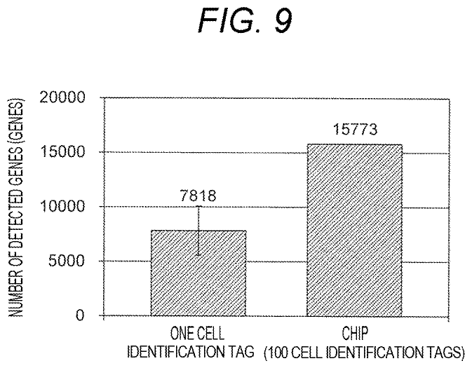

Analysis is performed with an NGS apparatus 128 using the final sample 127 obtained by introducing and capturing 80 cells in each chip 100 and undergoing various steps. That is, sequence reads thus obtained are separated using 100 kinds of the cell identification sequences 111, and then the number of genes detected in the sequence read of each of the cell identification sequences is investigated. As a result, three types of data of which continuities in the values of the number of detected genes are different from each other can be confirmed (FIG. 8). Specifically, two or three-cell data assuming capturing of the plurality of cells in each microreactor 103, single-cell data, and zero-cell data assuming no capturing of cells (various steps did not work well) can each be confirmed. An average number of detected genes in the single-cell data was 7,818, and a total number of detected genes per chip 100 was 15,773 (FIG. 9). In addition, in the present Example, Miseq (Illumina, Inc.) was used as the NGS apparatus, and an average number of reads per cell was as small as 0.14M reads, since throughput was 15M to 20M reads/run, which was rather low. It is considered that the number of detected genes per cell also approaches 15,773, which is the total number of detected genes per chip, in a case where the analysis is performed on the present sample using an NGS apparatus with throughput higher than 1G reads/run, and the number of reads per cell thus obtained is about 1M. From this, it was possible to verify that the comprehensive gene expression analysis on a single-cell level is possible. Furthermore, quantification precision was investigated by the method of the present Example, using ERCC (Ambion) obtained by mixing known amounts of 92 types of mRNA. As a result, an R2 value for data obtained from an amount equivalent to a single cell (0.614 pg) was 0.9073, and an R2 value for data obtained from an amount equivalent to 100 cells (61.4 pg) was 0.9714, and thus high quantification precision was verified (FIG. 7).

### EXAMPLE 2

As in Example 1, a method of this Example includes (1) a step of capturing cells on a chip in which microreactors are arranged in an array, (2) a step of capturing mRNA after cell lysis, (3) a step of synthesizing first cDNA on a surface of a support, (4) a step of pooling and washing the supports (magnetic beads) on which first cDNA libraries are immobilized by dispersing the supports in one tube, (5) a step of synthesizing second cDNA and performing washing, (6) a step of carrying out a tagmentation reaction and performing washing, (7) a PCR amplification step, and (8) an NGS analysis step. In the present Example, since an inexpensive reverse transcriptase is used in the step (3) of synthesizing first cDNA on a surface of a support instead of the expensive reverse transcriptase having a TS function used in Example 1, a cost of a reagent for preparing a sample can be reduced. Furthermore, since a random primer and a strand-displacement DNA polymerase are used in the step (5) of synthesizing second cDNA and performing washing, second cDNA synthesis efficiency is expected to be improved compared to that in Example 1. “(3) The step of synthesizing first cDNA on a surface of a support” and “(5) the step of synthesizing second cDNA and performing washing” that are different from those in Example 1 are described in detail below. Other steps are the same as those in Example 1.

(3) Step of Synthesizing First cDNA on Surface of Support: Use of Reverse Transcriptase not Having TS Function

After performing the step (1) of capturing cells on a chip in which microreactors are arranged in an array and the step (2) of capturing mRNA after cell lysis in the same manner as in Example 1, a cell lysis reagent is completely removed by increasing a negative pressure in a device. 2 μL of a cell washing solution (100 mM Tris (pH 8.0), 500 mM NaCl, and 5 mM DTT) is further added to the upper surfaces of all of the chips, and a negative pressure is immediately applied. Each microreactor is thoroughly washed by performing this operation twice, thereby removing the cell lysis reagent that can be an inhibitor for a subsequent reverse transcription reaction. 4 μL of a reverse transcription reaction reagent (1x FS buffer, 25 mM DTT, 2.5 mM dNTPs, 0.75% NP40, RNase OUT (4 U/μL), and SuperScript III (20 U/μL): Thermo Fisher Scientific Inc.) is added to the upper surfaces of all of the chips, the microreactors are filled with the reagent by applying a moderate negative pressure, and then the chips are incubated at 50° C. for 50 minutes. First cDNA 113 is thus synthesized in a 3′ direction of the probe 109 for a reverse transcription reaction, by using a captured mRNA molecule 108 as a template. Accordingly, the finally synthesized first cDNA has a sequence 112 for PCR amplification (SEQ ID NO: 4), a cell identification sequence 111 (SEQ ID NO: 3), and a molecule identification sequence 110 (SEQ ID NO: 2) on a 5′ end thereof. Through this step, the first cDNA libraries can be simultaneously synthesized from mRNA derived from all of genes that are expressed in a plurality of single cells, in a state of being immobilized on the supports.

(5) Step of Synthesizing Second cDNA and Performing Washing: Use of Random Primer and Strand-Displacement DNA Polymerase

After performing the step (4) of pooling and washing the supports (magnetic beads) on which the first cDNA libraries are immobilized by dispersing the supports into one tube in the same manner as in Example 1, the present sample is mixed with an Exonuclease I reagent (1x Buffer and Exonuclease I (1 U/μL)), and 5μL of the mixture is used as a reaction solution, which is then incubated at 37° C. for 15 minutes. The reaction solution is then incubated at 80° C. for 15 minutes in order to thermally deactivate Exonuclease I. The supports are washed twice with 50 μL of a washing solution (0.1% Tween 20 and 10 mM Tris (pH 8.0)). By this operation, a single-stranded probe 200 for a reverse transcription reaction which remains on the surface of the support without contributing to the synthesis of the first cDNA and can inhibit the synthesis of the second cDNA can be degraded and removed (FIG. 2A). Next, 5μL of an RNase H reagent (50 mM This-HCl (pH 8.3), 75 mM KCl, 3 mM MgCl₂, 20 mM DTT, and RNase H (1 U/μL): Thermo Fisher Scientific Inc.) is added to the same tube and mixed with the supports, and the mixture is incubated at 37° C. for 15 minutes. The supports are then washed twice with 50 μL of a washing solution (0.1% Tween 20 and 10 mM Tris (pH 8.0)). By this operation, mRNA 108 can be degraded and removed. Next, 5 μL of a second cDNA synthesis reagent (10 μM random primers 201 (SEQ ID NO: 13), 1x Bst Reaction Buffer, 0.25 mM dNTP mix, and Bst DNA polymerase (1.6 U/μL): NIPPON GENE CO., LTD.) is added to and mixed with the supports, and the mixture is incubated at 50° C. for 30 minutes. Since this reagent contains a strand-displacement DNA polymerase, complementary strand binding reactions proceed in succession so that strands (202 and 203) synthesized ahead, which start from the random primers 201 annealing to first DNA 113 at a plurality of sites thereof, are displaced (FIG. 2A). Complementary strands synthesized first fall off from the support and become by-products 205 present in the liquid phase, and second cDNA 204 which is a complementary strand synthesized by a random primer annealing near to a 3′ side of the first DNA 113 can be finally obtained in a state of being captured on the support. A supernatant which contains a residual reagent from the second cDNA synthesis reaction and the by-products 205 that are present in the liquid phase by falling off from the support is removed while the supports are captured by the neodymium magnet 118, and the supports are washed with 50 μL of a support washing solution (10 mM Tris and 0.1% Tween 20 (pH 8.0)).

The subsequent steps, (6) the step of carrying out a tagmentation reaction and performing washing, (7) the PCR amplification step, and (8) the NGS analysis step, are the same as those in Example 1.

### EXAMPLE 3

As in Examples 1 and 2, a method of this Example includes (1) a step of capturing cells on a chip in which microreactors are arranged in an array, (2) a step of capturing mRNA after cell lysis, (3) a step of synthesizing first cDNA on a surface of a support, (4) a step of pooling and washing the supports (magnetic beads) on which first cDNA libraries are immobilized by dispersing the supports into one tube, (5) a step of synthesizing second cDNA and performing washing, (6) a step of carrying out a tagmentation reaction and performing washing, (7) a PCR amplification step, and (8) an NGS analysis step. Since an inexpensive reverse transcriptase not having a TS function is used in the present Example as in Example 2, cost reduction is possible. Furthermore, second cDNA 209 is synthesized by synthesizing a complementary strand using a 5′-phosphorylated_3′-dideoxycytidine-modified oligo 207 (SEQ ID NO: 14) added by a single-stranded DNA ligase and a primer 208 (SEQ ID NO: 15), its complementary sequence (FIG. 2B). “(5) The step of synthesizing second cDNA and performing washing” that is different from that in Example 2 is described in detail below.

The step (1) of capturing cells on a chip in which microreactors are arranged in an array and the step (2) of capturing mRNA after cell lysis are performed in the same manner as in Examples 1 and 2. After performing the step (3)of synthesizing first cDNA on a surface of a support using the same method as that in Example 2, the step (4) of pooling and washing the supports (magnetic beads) on which first cDNA libraries are immobilized by dispersing the supports into one tube is performed in the same manner as in Examples 1 and 2.

(5) Step of Synthesizing Second cDNA and Performing Washing: Use of Single-Stranded DNA Ligase

In the same manner as in Example 2, the present sample is mixed with an Exonuclease I reagent (1x Buffer and Exonuclease I (1 U/μL): Takara Bio Inc.), and 5 μL of the mixture is used as a reaction solution, which is then incubated at 37° C. for 15 minutes. The reaction solution is then incubated at 80° C. for 15 minutes in order to thermally deactivate Exonuclease I. The supports are washed twice with 50 μL of a washing solution (0.1% Tween 20 and 10 mM Tris (pH 8.0)). By this operation, a single-stranded probe 200 for a reverse transcription reaction which remains on the surface of the support without contributing to the synthesis of the first cDNA and can inhibit the synthesis of the second cDNA can be degraded and removed (FIG. 2B). Next, 5 μL of an RNase H reagent (50 mM This-HCl (pH 8.3), 75 mM KCl, 3 mM MgCl₂, 20 mM DTT, and RNase H (1 U/μL): Thermo Fisher Scientific Inc.) is added to the same tube and mixed with the supports, and the mixture is incubated at 37° C. for 15 minutes. The supports are then washed twice with 50 μL of a washing solution (0.1% Tween 20 and 10 mM Tris (pH 8.0)). By this operation, degraded mRNA 206 can be removed. Next, 4 μL of a single-stranded DNA ligase reagent (1x Buffer, 50 μM dATP, 2.5 mM MgCl₂, Circ ssDNA Ligase (0.25 U/μL) 5′-phosphorylated_3′-dideoxycytidine-modified oligo 207 (SEQ ID NO: 14)) is added to the same tube and mixed with the supports, and the mixture is incubated at 60° C. for 1 hour and then at 80° C. for 10 minutes. The 5′-phosphorylated_3′-dideoxycytidine-modified oligo 207 is added to a 3′ end of the first cDNA by this reaction. Next, 5 μL of a second cDNA synthesis reagent (1x Tks Gflex Buffer, Tks Gflex DNA polymerase (0.125 U/μL: Takara Bio Inc.), and 6 μM primers 208 for second cDNA synthesis (SEQ ID NO: 15)) is mixed with the supports in the same tube, and a reaction is carried out using a thermal cycler with the following temperature condition: 98° C. for 1 minute, 50° C. for 5 minutes, and 68° C. for 6 minutes, thereby synthesizing the second cDNA 209 (FIG. 2B). A supernatant which contains a residual reagent from the second cDNA synthesis reaction is removed while the supports are captured by the neodymium magnet 118, and the supports are washed with 50 μL of a support washing solution (10 mM Tris and 0.1% Tween 20 (pH 8.0)).

The subsequent steps, (6) the step of carrying out a tagmentation reaction and performing washing, (7) the PCR amplification step, and (8) the NGS analysis step, are the same as those in Example 1.

### EXAMPLE 4

As in Examples 1 to 3, a method of this Example includes (1) a step of capturing cells on a chip in which microreactors are arranged in an array, (2) a step of capturing mRNA after cell lysis, (3) a step of synthesizing first cDNA on a surface of a support, (4) a step of pooling and washing the supports (magnetic beads) on which first cDNA libraries are immobilized by dispersing the supports into one tube, (5) a step of synthesizing second cDNA and performing washing, (6) a step of carrying out a tagmentation reaction and performing washing, (7) a PCR amplification step, and (8) an NGS analysis step. Since an inexpensive reverse transcriptase not having a TS function is used in the present Example as in Examples 2 and 3, cost reduction is possible. Second cDNA 212 is synthesized by adding continuous nucleotides(N) (a poly T sequence in the present Example) 210 to a 3′ end of the first cDNA using a terminal transferase and synthesizing a complementary strand using a primer of its complementary sequence (a poly A sequence in the present Example) 211 (SEQ ID NO: 16) (FIG. 2C). “(5) The step of synthesizing second cDNA and performing washing” that is different from those in Examples 2 and 3 is described in detail below.

The step (1) of capturing cells on a chip in which microreactors are arranged in an array and the step (2) of capturing mRNA after cell lysis are performed in the same manner as in Examples 1 to 3. After performing the step (3) of synthesizing first cDNA on a surface of a support using the same method as that in Example 2, the step (4) of pooling and washing the supports (magnetic beads) on which first cDNA libraries are immobilized by dispersing the supports into one tube is performed in the same manner as in Examples 1 to 3.

(5) Step of Synthesizing Second cDNA and Performing Washing: Use of Terminal Transferase

In the same manner as in Examples 2 and 3, the present sample is mixed with an Exonuclease I reagent (1x Buffer and Exonuclease I (1 U/μ): Takara Bio Inc.), and 5 μL of the mixture is used as a reaction solution, which is then incubated at 37° C. for 15 minutes. The reaction solution is then incubated at 80° C. for 15 minutes in order to thermally deactivate Exonuclease I. The supports are washed twice with 50 μL of a washing solution (0.1% Tween 20 and 10 mM Tris (pH 8.0)). By this operation, a single-stranded probe 200 for a reverse transcription reaction which remains on the surface of the support without contributing to the synthesis of the first cDNA and can inhibit the synthesis of the second cDNA can be degraded and removed (FIG. 2C). Next, 5 μL of an RNase H reagent (50 mM This-HCl (pH 8.3), 75 mM KCl, 3 mM MgCl₂, 20 mM DTT, and RNase H (1 U/μL): Thermo Fisher Scientific Inc.) is added to the same tube and mixed with the supports, and the mixture is incubated at 37° C. for 15 minutes. The supports are then washed twice with 50 μL of a washing solution (0.1% Tween 20 and 10 mM Tris (pH 8.0)). By this operation, degraded mRNA 206 (FIG. 2C) can be removed. 12 μL of a transferase reaction solution (5 mM Tris-HCl (pH 8.3), 25 mM KCl, 0.75 mM MgCl₂, 1.5 mM dATP, RNase H (0.15 U/μL), a terminal transferase (0.188 U/μL), and 0.45% NP40) is added to the same tube and mixed with the supports, and then the mixture is incubated at 30° C. for 15 minutes and then at 70° C. for 5 minutes. The supports are washed twice with 50 μL of a washing solution (0.1% Tween 20 and 10 mM Tris (pH 8.0)). The continuous nucleotides(N) (a poly T sequence in the present Example) 210 are added to the 3′ end of the first cDNA by this reaction. Next, 5 μL of a second cDNA synthesis reagent (1x Tks Gflex Buffer, Tks Gflex DNA polymerase (0.125 U/μL), and 1 μM 3′-end-BN-addedpoly_A-sequence primers 211 (SEQ ID NO: 16): Takara Bio Inc.) is mixed with the supports in the same tube, and a reaction is carried out using a thermal cycler with the following temperature condition: 98° C. for 1 minute, 44° C. for 5 minutes, and 68° C. for 6 minutes, thereby synthesizing the second cDNA 212 (FIG. 2C). A supernatant which contains a residual reagent from the second cDNA synthesis reaction is removed while the supports are captured by the neodymium magnet 118, and the supports are washed with 50 μL of a support washing solution (10 mM Tris and 0.1% Tween 20 (pH 8.0)).

The subsequent steps, (6) the step of carrying out a tagmentation reaction and performing washing, (7) the PCR amplification step, and (8) the NGS analysis step, are the same as those in Example 1.

### EXAMPLE 5

As in Examples 1 to 4, a method of this Example includes (1) a step of capturing cells on a chip in which microreactors are arranged in an array, (2) a step of capturing mRNA after cell lysis, (3) a step of synthesizing first cDNA on a surface of a support, (4) a step of pooling and washing the supports (magnetic beads) on which first cDNA libraries are immobilized by dispersing the supports into one tube, (5) a step of synthesizing second cDNA and performing washing, (6) a step of carrying out a tagmentation reaction and performing washing, (7) a PCR amplification step, and (8) an NGS analysis step. However, in the present Example, a chip including a microreactor 103 filled with a support on which a random primer 213 is immobilized in addition to a probe 109 for a reverse transcription reaction is used (FIG. 3A and FIG. 3B). Furthermore, since an inexpensive reverse transcriptase not having a TS function is used as in Examples 2 to 4, cost reduction is possible.

The step (1) of capturing cells on a chip in which microreactors are arranged in an array and the step (2) of capturing mRNA after cell lysis are performed in the same manner as in Example 1, except that the support on which the random primer (another sequence for PCR may be added to a 5′ side thereof) 213 is immobilized in addition to the probe 109 for a reverse transcription reaction (SEQ ID NO: 1) is used. After performing the step (3) of synthesizing first cDNA on a surface of a support (FIG. 3A) using the same method as those in Examples 2 to 4, the step (4) of pooling and washing the supports (magnetic beads) on which first cDNA libraries are immobilized by dispersing the supports into one tube is performed in the same manner as in Example 1. Next, 5 μL of an RNase H reagent (50 mM This-HCl (pH 8.3), 75 mM KCl, 3 mM MgCl₂, 20 mM DTT, and RNase H (1 U/μL,): Thermo Fisher Scientific Inc.) is added to the same tube and mixed with the supports, and the mixture is incubated at 37° C. for 15 minutes. The supports are then washed twice with 50 μL of a washing solution (0.1% Tween 20 and 10 mM Tris (pH 8.0)). By this operation, mRNA 108 can be degraded and removed (FIG. 3A). “(5) The step of synthesizing second cDNA and performing washing” that is different from those in Examples 1 to 4 is described in detail below.

(5) Step of Synthesizing Second cDNA and Performing Washing: Use of Immobilized Random Primer and Strand-Displacement DNA Polymerase

5 μL of a second cDNA synthesis reagent (1x Bst Reaction Buffer, 0.25 mM dNTP mix, and Bst DNA polymerase (1.6 U/μL: NIPPON GENE CO., LTD.)) containing a strand-displacement DNA polymerase is added to the same tube and mixed with the supports, and the mixture is incubated at 50° C. for 30 minutes. In this step, a portion of a sequence of first DNA 113 that is complementary to the random primer 213 immobilized on the support anneals to the random primer 213, and second cDNA 214 is synthesized (FIG. 3B). That is, a second cDNA strand is obtained in a state of being immobilized on the support as well, unlike those in Examples 1 to 4. Next, a portion of the first cDNA that is complementary to another random primer anneals to the random primer, and a new second cDNA 215 can be synthesized. A plurality of second cDNA molecules are synthesized from one molecule of the first cDNA in this manner, and the amplified second cDNA molecules can further anneal to the probe 109 for a reverse transcription reaction (SEQ ID NO: 1) on the support, whereby a new cDNA strand can be synthesized. That is, cDNA derived from a single cell can be amplified in a state of being captured on the support (FIG. 3B). In this manner, detection sensitivity and quantification precision can be improved even in a case of a low-expression gene. Next, a supernatant which contains a residual reagent from the second cDNA synthesis reaction and a by-product 205 that is present in the liquid phase by falling off from the support is removed while the supports are captured by a neodymium magnet 118, and the supports are washed with 50 μL of a support washing solution (10 mM Tris and 0.1% Tween 20 (pH 8.0)).

The subsequent steps, (6) the step of carrying out a tagmentation reaction and performing washing, (7) the PCR amplification step, and (8) the NGS analysis step, are the same as those in Example 1.

### REFERENCE SIGNS LIST

100 chip

101 cell

102 micro through-hole

103 microreactor

104 support

105 reagent discharge part

106 lysed cell membrane

107 lysed nuclear membrane

108 mRNA

109 probe for reverse transcription reaction

110 molecule identification sequence

111 cell identification sequence

112 sequence for PCR amplification

113 first DNA

114 TS specific sequence added by reverse transcriptase having template switch (TS) function

115 SMART-Seq v4 Oligo

116 PCR tube

117 buffer for dispersing supports

118 neodymium magnet

119 primer for second cDNA synthesis

120 second cDNA

121 common sequence portion (19 bases)

122 specific sequence A portion (14 bases)

123 specific sequence B portion (15 bases)

124 sequence for NGS analysis (P5_R1SP)

125 sequence for NGS analysis (P7_R2SP)

126 chip identification sequence

127 final sample

128 NGS analysis apparatus

130 porous membrane

200 single-stranded probe 109 for reverse transcription reaction degraded by Exonuclease I

201 random primer

202 strand annealing to first cDNA 113 before 201 and subjected to complementary strand elongation reaction by strand-displacement DNA polymerase

203 strand annealing to first cDNA 113 before 201 and 202 and subjected to complementary strand elongation reaction by strand-displacement DNA polymerase

204 second cDNA immobilized on support

205 by-product that is present in liquid phase by falling off from support

206 mRNA degraded by RNase

207 5′ -phosphorylated_3′-dideoxycytidine-modified oligo added by single-stranded DNA ligase

208 primer for second cDNA synthesis having sequence complementary to 207

209 second cDNA synthesized using 208

210 poly T sequence added by terminal transferase

211 poly A sequence primer having BN added to 3′ end thereof

212 second cDNA synthesized using poly A sequence primer 211

213 random primer immobilized on support

214 second cDNA immobilized on support

215 second cDNA synthesized by annealing of another random primer on 3′ end side of first cDNA

### SEQUENCE LISTING FREE TEXT

All of the sequences shown below are artificial oligonucleotides and are shown in a 5→3′ direction.

SEQ ID NO: 1: probe 109 for a reverse
transcription reaction (showing an example among
100 kinds of cell identification tags)
CCATCTCATCCCTGCGTGTCTCCGACTCAGCGTACTNNNNNNNTTTTTTT
TTTTTTTTTTTVN
SEQ ID NO: 2: molecule identification sequence
110 (N = A, G, C, or T)
NNNNNNN
SEQ ID NO: 3: cell identification sequence 111
(showing an example among 100 kinds of known
Sequences)
CGTACT
SEQ ID NO: 4: sequence 112 for PCR amplification
CCATCTCATCCCTGCGTGTCTCCGACTCAG
SEQ ID NO: 5: common Sequence portion 121
AGATGTGTATAAGAGACAG
SEQ ID NO: 6: specific Sequence A portion 122
TCGTCGGCAGCGTC
SEQ ID NO: 7: specific sequence B portion 123
GTCTCGTGGGCTCGG
SEQ ID NO: 8: Sequence 124 for NGS
analysis (P5_R1SP)
AATGATACGGCGACCACCGAGATCTACACTCTTTCCCTACACGACGCTCT
TCCGATCT
SEQ ID NO: 9: sequence 125 for NGS
analysis (P7_R2SP)
CAAGCAGAAGACGGCATACGAGATGTGACTGGAGTTCAGACGTGTGCTCT
TCCGATCT
SEQ ID NO: 10: chip identification Sequence 126
(showing an example among 16 kinds)
CGATA
SEQ ID NO: 11: P5
AATGATACGGCGACCACCGAGATCTACAC
SEQ ID NO: 12: P7
CAAGCAGAAGACGGCATACGAGAT
SEQ ID NO: 13: random primer 201
(N = A, G, C, or T)
NNNNNNNNN
SEQ ID NO: 14: 5′-phosphorylated_3′-
dideoxycytidine-modified oligo 207
(5′P)AGCAACGCACTTTGAATTTTGTAATCCTGAAGGG(3′ddC)
SEQ ID NO: 15: primer 208 for second cDNA
synthesis having a sequence complementary
to 207
CCCTTCAGGATTACAAAATTCAAAGTGCGTTGCT
SEQ ID NO: 16: poly A sequence primer 211 having
BN added to the 3′ end thereof (B = G, C, or T
and N = A, G, C, or T)
AAAAAAAAAAAAAAAAAAAAAAAAABN

1. A method for analyzing gene expression in a cell using a device comprising a plurality of microreactors, wherein the microreactores are filled with one or more solid supports on which a probe having a primer sequence for amplification, a cell identification sequence, a molecule identification sequence, and an oligo (dT) sequence is immobilized, and the method comprises:
a step of introducing a plurality of cells into the microreactors so that a single cell corresponds to each one of the microreactors;
a step of capturing mRNA derived from the single cell on the probe;
a step of synthesizing first cDNA by subjecting the captured mRNA to a reverse transcription reaction, to produce a first cDNA library derived from the single cell on the solid supports;
a step of washing the solid supports;
a step of synthesizing second cDNA from the first cDNA library;
a step of performing fragmentation of double-stranded DNA comprising the first cDNA and the second cDNA and addition of a tag sequence;
a step of removing a component other than an immobilized double-stranded DNA fragment by washing the solid supports with a washing solution;
a step of performing amplification of the double-stranded DNA fragment using a primer having at least a portion of the primer sequence for amplification and the tag sequence or a sequence complementary to at least a portion of the primer sequence for amplification and the tag sequence to amplify only a sequence derived from a 3′ end sequence of the mRNA; and
a step of performing, for the amplified sequence, analysis of gene expression in each single cell, using the cell identification sequence and the molecule identification sequence.

2. The method according to claim 1, wherein
the tag sequence comprises a specific sequence portion and a common sequence portion, and
the common sequence in the tag sequence or a sequence complementary to the common sequence is amplified in the amplification step.

3. The method according to claim 1, further comprising, before or after the step of washing the solid supports:
a step of pooling the solid supports, on which the first cDNA libraries derived from the single cells are immobilized, corresponding to the plurality of cells.

4. The method according to claim 3, wherein the solid supports, on which the first cDNA libraries derived from the single cells are immobilized, corresponding to 10 to 100,000 cells are pooled.

5. The method according to claim 1, wherein the solid support has a diameter of 10 nm to 100 μm.

6. The method according to claim 1, wherein the solid support is a magnetic bead.

7. The method according to claim 1, wherein the reverse transcription reaction is carried out using a reverse transcriptase having a template switch function.

8. The method according to claim 1, wherein the second cDNA is synthesized in the step of synthesizing the second cDNA by carrying out a complementary strand elongation reaction using a random primer and a DNA polymerase having a strand displacement activity.

9. The method according to claim 7, wherein the second cDNA is synthesized in the step of synthesizing the second cDNA by carrying out a complementary strand elongation reaction using a primer having a sequence complementary to a specific sequence added by the reverse transcriptase having the template switch function.

10. The method according to claim 1, wherein the second cDNA is synthesized in the step of synthesizing the second cDNA by adding a known sequence to a 3′ end of the first cDNA using a single-stranded DNA ligase and carrying out a complementary strand elongation reaction using a primer having a sequence complementary to the known sequence.

11. The method according to claim 1, wherein the second cDNA is synthesized in the step of synthesizing the second cDNA by adding a polyN sequence, which is a poly T, A, G, or C sequence, to a 3′ end of the first cDNA using a terminal transferase (TdT) and carrying out a complementary strand elongation reaction using a primer having a sequence complementary to the polyN sequence.

12. The method according to claim 1, wherein
the probe having the primer sequence for amplification, the cell identification sequence, the molecule identification sequence, and the oligo (dT) sequence, and a primer having a random sequence are immobilized on the solid supports, and
the second cDNA is synthesized in the step of synthesizing the second cDNA by carrying out a complementary strand elongation reaction using the random primer immobilized on the solid supports and a DNA polymerase having a strand displacement activity, to amplify cDNA.

13. The method according to claim 1, wherein
a through-hole having a diameter of 10 μm or smaller is formed in each microreactor, and
the single cell is captured on the through-hole in the cell introduction step.

14. The method according to claim 1, wherein the step of performing the fragmentation of the double-stranded DNA and the addition of the tag sequence is performed by subjecting the double-stranded DNA to a tagmentation reaction.
